SEQUENCE LISTING (1 sequence)

SEQ ID NO: 1 - protein, 441 aa, Homo sapiens
MKTLPLHSHCTEMQRLLPKLEMLTLGSSFCSLQGEFCQAMDSFTTVSHVGMCEETDASFNVFSPKFQFDRSHCQSLRFHQNMELDFGHFDERDKTSRNMRGSRMNGLPSPTHSAHCSFYRTRTLQALSNEKKAKKVRFYRNGDRYFKGIVYAVSSDRFRSFDALLADLTRSLSDNINLPQGVRYIYTIDGSRKIGSMDELEEGESYVCSSDNFFKKVEYTKNVNPNWSVNVKTSANMKAPQSLASSNSAQARENKDFVRPKLVTIIRSGVKPRKAVRVLLNKKTAHSFEQVLTDITEAIKLETGVVKKLYTLDGKQVTCLHDFFGDDDVFIACGPEKFRYAQDDFSLDENECRVMKGNPSATAGPKASPTPQKTSAKSPGPMRRSKSPADSANGTSSSQLSTPKSKQSPISTPTSPGSLRKHKDLYLPLSLDDSDSLGDSM

(19) United States
(12) Patent Application Publication
(10) Pub. No.: US 2006/0257949 A1
(43) Pub. Date: Nov. 16, 2006

(54) Methods of diagnosing and treating brain tumors

(76) Inventors: Alonzo H. Ross, Shrewsbury, MA (US); Marie-Claire Daou, Worcester, MA (US); Thomas Smith, Holden, MA (US); N. Scott Litofsky, Columbia, MO (US); Chung Hsieh, Wellesley, MA (US)

Correspondence Address: FISH & RICHARDSON PC, P.O. BOX 1022, MINNEAPOLIS, MN, 55440-1022, US

(21) Appl. No.: 11/166,894

(22) Filed: Jun. 24, 2005

Related U.S. Application Data
(60) Provisional application No. 60/582,873, filed on Jun. 24, 2004.

(51) Int. Cl. G01N 33/574

(52) U.S. Cl. 435/7.23

(57) ABSTRACT

The invention relates to methods and compositions for the diagnosis and treatment of brain tumors.

### CLAIM OF PRIORITY

This application claims priority under 35 USC § 119(e) to U.S. Provisional Application No. 60/582,873, filed on Jun. 24, 2004, the entire contents of which are hereby incorporated by reference.

### BACKGROUND

Human brain tumors are classified as primary (arising from cells intrinsic to the brain and its coverings) or secondary (metastatic). One category of primary brain tumors is derived from neuroepithelial cells (i.e., glial or neuronal cells) and their primitive precursor cells. Tumors composed of glial cells are collectively known as gliomas, and are classified by their predominant cell type as determined by conventional morphologic, ultrastructural, and/or immunohistochemical features. The major categories of gliomas are astrocytoma (resembling astrocytes), oligodendroglioma (resembling oligodendrocytes), mixed glioma (at least two lineages, i.e., astrocytic, oligodendroglial and/or ependymal), and ependymoma (resembling ependymal cells). Gliomas can also be assessed for their grade or degree of anaplasia (malignancy) using histologic parameters such as cytologic atypia, mitotic activity, vascular proliferation, and necrosis. The most commonly used grading scheme is the 4-tier World Health Organization (WHO) system. Most gliomas are invasive, including some low-grade tumors (Giese et al., J. Clin. Oncol., 21:1624-36 (2003); Cavenee, W. K. et al, Diffuse Astrocytomas, in Pathology and Genetics of Tumours of the Nervous System, International Agency for Research on Cancer (1997)). The major exceptions are the pilocytic astrocytoma (WHO grade I), which shows little or no tendency to invade brain, and ependymomas, which are likewise minimally invasive but sometimes can spread through the cerebrospinal fluid.

### SUMMARY

The present invention is based, at least in part, on the discovery that the protein doublecortin is expressed in invasive brain tumors, and that doublecortin expression is correlated with brain tumor invasiveness. Thus, the present invention features methods of diagnosing and treating brain tumors, and also features methods of screening for compounds for the treatment of brain tumors, based on the detection or modulation of doublecortin.

Accordingly, in one aspect, the present invention provides methods for diagnosing a brain tumor in a subject. The methods include obtaining a biological sample from the brain or cerebrospinal fluid of the subject and determining the level or activity of doublecortin in the biological sample. The biological sample can be, e.g., a biopsy, or a sample of cerebrospinal fluid. The methods further include comparing the level or activity of doublecortin in the biological sample to a control sample, e.g., a control biological sample from a subject not having a brain tumor. If a doublecortin activity or level in the biological sample is elevated compared to that in the control sample, the subject can be diagnosed as having a brain tumor. Doublecortin level or activity can be assessed using methods known in the art, including, but not limited to, detecting doublecortin protein using, e.g., an anti-doublecortin antibody, e.g., a labeled anti-doublecortin antibody, or detecting doublecortin mRNA using, e.g., a nucleic acid probe, e.g., a labeled nucleic acid probe.

In certain embodiments, the methods further include determining the level or activity of doublecortin and comparing the level or activity to a threshold level, wherein a level or activity above the threshold level indicates that the brain tumor is an invasive brain tumor. The threshold level can be, e.g., the level of doublecortin expression or activity in a control biological sample from a subject not having a brain tumor, or the level of doublecortin expression or activity in a control biological sample from a non-invasive brain tumor.

In another aspect, the invention features methods of treating a subject having or at risk for developing an invasive brain tumor by administering to the subject a therapeutically effective amount of a compound, e.g., a compound identified by a method described herein, that selectively reduces the level or activity of doublecortin. In some embodiments, a therapeutically effective amount of a composition is administered that includes one or more compounds selected from the group consisting of a small molecule, peptide, nucleic acid molecule, or polypeptide, e.g., a doublecortin-interacting protein, that modulate doublecortin level or activity. In some embodiments, the compound is an antisense nucleic acid, an siRNA, or a ribozyme that selectively binds to a nucleic acid, e.g., an mRNA, encoding doublecortin.

As used here, the term “therapeutically effective amount” refers to an amount of a compound or composition sufficient to inhibit or stop growth, or to inhibit or slow cellular proliferation, of an existing tumor (or to cause the size of the tumor to decrease) or to inhibit the formation of an invasive brain tumor in a subject at risk for developing a tumor.

In other embodiments, the methods further include identifying a subject having or at risk for developing an invasive brain tumor. The methods can include obtaining a biological sample from the brain or cerebrospinal fluid from the subject, determining the level or activity of doublecortin in the biological sample, and comparing the level or activity to a threshold level, wherein a level or activity above the threshold level indicates that the subject has or is at risk for an invasive brain tumor. The threshold level can be, e.g., the level of doublecortin expression or activity in a control biological sample from a subject not having a brain tumor, or the level of doublecortin expression or activity in a control biological sample from a non-invasive brain tumor.

In another aspect, the invention features methods of identifying a candidate compound for the treatment of a brain tumor, e.g., an invasive brain tumor. The method includes (a) contacting a doublecortin polypeptide with a test compound; and (b) detecting an interaction (e.g., specific binding) of the test compound with the doublecortin polypeptide, wherein an interaction indicates that the test compound is a candidate compound for the treatment of an invasive brain tumor. Optionally, the method can further include (c) determining whether the candidate compound modulates doublecortin activity in vivo or in vitro, wherein modulation indicates that the candidate compound is a doublecortin modulating agent. In other embodiments, the test compound is immobilized on a substrate, and interaction of the candidate compound with the polypeptide is detected as immobilization (indicating specific binding) of the polypeptide on the immobilized test compound. In still other embodiments, the test compound is a small molecule, peptide, nucleic acid molecule, or polypeptide.

In yet another aspect, the invention includes methods for identifying a candidate compound capable of modulating doublecortin level or activity by (a) contacting a nucleic acid encoding doublecortin with a test compound; and (b) detecting an interaction of the test compound with the nucleic acid, wherein an interaction indicates that the test compound is a candidate compound. Optionally, the method can include (c) determining whether a candidate compound inhibits doublecortin activity in vivo, relative to doublecortin activity in the absence of the test compound that interacts with the nucleic acid, wherein inhibition of doublecortin activity in vivo indicates that the candidate compound is a doublecortin modulating agent. In certain embodiments, the test compound is a small molecule, peptide, nucleic acid molecule, or polypeptide.

In another aspect, the invention includes pharmaceutical compositions that include a candidate compound and/or doublecortin modulating agent, e.g., identified by a method described herein and a pharmaceutically acceptable excipient.

“Gene” refers to a DNA sequence that encodes through its template or messenger RNA a sequence of amino acids characteristic of a specific polypeptide. The term “gene” includes intervening, non-coding regions, as well as regulatory regions, and can include 5′ and 3′ ends.

As used herein, the terms “nucleic acid,” “nucleic acid molecule,” “nucleotide sequence,” and “oligonucleotide” include single-stranded and double-stranded nucleic acids including, but not limited to, DNAs, RNAs (e.g., mRNA, tRNAs, rRNAs, siRNAs, and shRNAs), cDNAs, recombinant DNA (rDNA), antisense nucleic acids, oligonucleotides, oligomers, and polynucleotides. The terms encompass both sense and antisense strands unless otherwise noted. The terms may also include hybrids such as triple-stranded regions of RNA and/or DNA or double-strand RNA:DNA hybrids. The terms are also contemplated to include modified nucleic acids, e.g., biotinylated nucleic acids, tritylated nucleic acids, fluorophore labeled nucleic acids, inosine, and the like.

The nucleic acids described herein can be derived from a variety of sources, including DNA, cDNA, synthetic DNA, synthetic RNA, or combinations thereof. Such sequences can comprise genomic DNA that may or may not include naturally occurring introns. Moreover, such genomic DNA may be associated with promoter regions and/or poly(A) sequences. The sequences, genomic DNA or cDNA may be obtained in any of several ways. Genomic DNA can be extracted and purified from suitable cells by means well-known in the art. Alternatively, mRNA can be isolated from a cell and used to produce cDNA by reverse transcription or other means. An “isolated gene” or “isolated nucleic acid” excludes genomic sequences found upstream or downstream of a gene.

“Expression” refers to the transcription of a gene sequence and subsequent processing steps, such as translation, of a resultant mRNA to produce the final end product of a gene. The end product may be a protein (such as an enzyme or receptor) or a nucleic acid (such as tRNA, antisense RNA, or other regulatory factor).

A “promoter region” includes a promoter as well as other sequences necessary for the initiation of transcription of a gene. The presence of a promoter region is sufficient to cause the expression of a gene sequence operably linked to the promoter region. A “promoter” is a DNA sequence located 5′ to a gene that can be recognized by an RNA polymerase and indicates the site for transcription initiation. The presence of such a sequence permits the RNA polymerase to bind to and initiate transcription of operably linked gene sequences. Many different promoters are known in the art that direct expression of a gene in a certain cell type. Tissue-specific promoters can comprise nucleic acid sequences that cause a greater (or decreased) level of expression in cells of a certain tissue type. Tissue-specific promoters also encompass “leaky” promoters, which regulate expression of a selected nucleic acid primarily in one tissue, but can cause at least low level expression in other tissues as well. A promoter is “operably linked” to a sequence of DNA if upon introduction into a host cell the promoter controls the transcription of the DNA sequence(s) into one or more species of RNA.

A “vector” is a nucleic acid molecule capable of transporting another nucleic acid to which it has been linked. Vectors capable of directing the expression of nucleic acids to which they are linked are referred to herein as “expression vectors.” In general, expression vectors of utility in recombinant DNA techniques are often in the form of plasmids, which are generally circular double-stranded DNA not bound to a chromosome. The present methods and compositions encompass other forms of expression vectors that serve equivalent functions. Numerous expression vectors are known in the art and can be used to express the polynucleotides and/or polypeptides described herein.

An “antibody” is a polyclonal, monoclonal and/or monospecific antibody and fragments thereof, and antigen-binding fragments thereof, that can bind to the doublecortin protein and fragments thereof. The term antibody is used to refer both to a homogeneous molecular entity, or a mixture such as a serum product made up of a plurality of different molecular entities. Methods for making antibodies are known in the art. The term “antibody” is meant to include, but is not limited to, a polyclonal, monoclonal, chimeric, human, humanized, bispecific, multispecific, and a Primatized® antibody, and includes a synthetically or recombinantly produced molecule that binds to a target protein. Antigen-binding fragments include single chain antibodies (ScFv), Fab and F(ab)₂.

By “modulate” is meant to increase or decrease the wild-type activity or binding of an enzyme or other protein. Modulation can be effected by affecting the concentration or subcellular localization of a biologically active protein, i.e., by regulating expression or degradation, or by direct agonistic or antagonistic effect, e.g., through inhibition, activation, binding, or release of binding partners, modification (either chemically or structurally) or by direct or indirect interaction that can involve additional factors. Modulated activities of doublecortin can include binding to other proteins, such as doublecortin-binding proteins, and binding to antibodies.

Substantially complementary oligonucleotide sequences are greater than about 80 percent complementary to the full length of the corresponding target sequence to which the oligonucleotide binds. In some embodiments, the substantially complementary oligonucleotide sequences will be greater than about 85%, 90%, 95%, 96%, 97%, 98%, or 99% complementary to the corresponding target sequence, or will be 100% complementary to the corresponding target sequence.

Substantially identical oligonucleotide or amino acid sequences are greater than about 90 percent identical to a reference sequence. In some embodiments, the identical oligonucleotide sequences will be greater than about 95%, 96%, 97%, 98%, or 99% identical, or will be 100% identical, to the reference sequence.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a micrograph of anti-doublecortin immunostaining of a 5 μM section of a human glioblastoma. FIG. 1B is anti-doublecortin immunostaining of a 5 μm section of a human oligoastrocytoma. FIG. 1C is anti-doublecortin immunostaining of a 5 μM section of a human pilocytic astrocytoma. Bar=30 μm.

FIG. 2 is a comparison of doublecortin expression in three tumor groups.

FIG. 3 is the amino acid sequence of a splice variant of human doublecortin (see GenBank Accession Number NP_{—}000546).

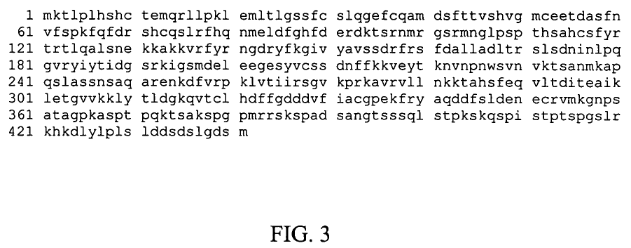

### DETAILED DESCRIPTION

The present invention is based, at least in part, on the discovery that doublecortin expression is correlated with brain tumor invasiveness. Thus, the present invention features methods of diagnosing brain tumors based on the detection of doublecortin and also features methods of treating brain tumors by modulating doublecortin level or activity. In addition, the invention features methods of screening test compounds to discover candidate compounds and agents for the treatment of brain tumors by screening for compounds that modulate doublecortin level or activity.

Doublecortin

Doublecortin (DCX) is a microtubule-associated phosphoprotein maintaining cytoskeletal plasticity during axonal outgrowth, neuronal maturation and cell migration (Horesh et al., Hum. Mol. Genet., 8:1599-1610 (1999)). DCX physically associates with microtubules (Francis et al., Neuron, 23:247-256 (1999); Gleeson et al., Neuron, 23:257-271 (1999) and is both a microtubule-stabilizing agent and a microtubule-bundling protein (Gleeson et al., supra; Horesh et al., supra). Impaired DCX function in vitro results in interruption of microtubuli. DCX also binds to the phosphorylated from of the L1 family member neurofascin (Kizhatil et al., J. Neurosci., 22:7948-7958 (2002)), which is a cell adhesion molecule, suggesting that DCX serves both as a regulator of microtubule polymerization and as a bridge between the microtubule cytoskeleton and the membrane-organizing protein, neurofascin. Thereby, the cellular shape and cytoskeletal function, as well as cell migration, are compromised in vivo (Gleeson et al., Cell, 92:63-72 (1998); Gleeson et al., Neuron, 23:257-271 (1999); Sapir et al., Hum. Mol. Genet., 9:703-712 (2000)) by impaired DCX function.

During development of the cortex, cells in the ventricular zone that express tubulin βIII and, therefore, have committed to a neuronal lineage, also express DCX (Francis et al., Neuron, 23:247-56 (1999); Gleeson et al, Neuron, 23:257-71(1999)). DCX is expressed by actively migrating neuronal precursor cells and post-migratory neurons at the cortical plate as they differentiate and extend neurites. In the developing retina, DCX has a similar expression pattern (Lee et al., Eur. J. Neurosci., 17:1542-8 (2003)). In adults, neurogenesis is confined to a few sites, such as the subventricular zone (SVZ). There is a clear population of DCX-positive cells in the SVZ and in the rostral migratory stream along which neuronal precursor cells migrate to the olfactory bulb (Gleeson et al., supra; Nacher et al., Eur. J. Neurosci., 14:629-44 (2001)).

Lissencephaly is a cerebral developmental disorder. Patients with lissencephaly (which means smooth brain (Feng et al., Nat. Rev. Neurosci., 2:408-416 (2001); Olson et al., Curr. Opin. Genet. Dev., 12:320-327 (2002)) are born with a thick, disorganized cortex, lacking gyri and folds. These patients suffer from severe mental retardation and epilepsy. The autosomal dominant form of this disease is caused primarily by mutations in the Lissencephaly-1 (LIS1) gene (Vallee et al., Trends Cell. Biol., 11:155-160 (2001)). The chromosome X-linked form of lissencephaly is due to mutations in the doublecortin (DCX) gene. In both cases, the mutations lead to reduced migration of neuronal precursor cells (Kato et al., Hum. Mol. Genet., 12:R89-96 (2003)). Recently, in a rat model, inhibition of DCX expression with RNAi vectors impaired radial migration of precursors in the cortex, mimicking human lissencephaly (Bai et al., Nat. Neurosci., 6:1277-1283 (2003)).

DCX is expressed in gangliogliomas (Becker et al., Acta Neuropathol. (Berl), 104:403-408 (2002)) and cortical tubers (Lee et al., Ann. Neurol., 53:668-673 (2003)). Also, in a lissencephaly subset with cerebellar hypoplasia, the large dysplastic neurons are DCX-positive (Miyata et al., Acta Neuropathol. (Berl), 107:69-81 (2004)). The role of DCX in these neuropathologies is not known.

General Methodology

As described herein, doublecortin is expressed in invasive brain tumors, and the expression of doublecortin is correlated with brain tumor invasiveness. Thus, the present invention relates, in part, to methods of diagnosing brain tumors involving detecting doublecortin protein or mRNA levels in a biological sample, e.g., brain tissue or cerebrospinal fluid.

The present invention also features methods of treating invasive brain tumors by targeting doublecortin level or activity with a therapeutic compound that reduces doublecortin level or activity. Methods of reducing doublecortin expression can include, e.g., RNA interference, antisense oligonucleotides, morpholino oligonucleotides, and immunoglobulins or fragments thereof that bind to doublecortin.

The present invention also features methods of modulating doublecortin level or activity, methods of identifying test compounds and compositions that modulate doublecortin level or activity, and compounds and compositions identified by the methods described herein. The present methods are useful in identifying compounds that can be used to treat and/or inhibit formation of an invasive brain tumor, and to identify novel targets for compounds that can be used to treat and/or inhibit formation of an invasive brain tumor.

Alternative embodiments provide methods for screening candidate drugs and therapies directed to treating and/or inhibiting formation of invasive brain tumors, and for identifying novel doublecortin-interacting proteins that are targets for drugs and therapies for treating and/or inhibiting formation of invasive brain tumors.

Methods of Diagnosing Invasive Brain Tumors

The invention is based, in part, on the observation that doublecortin is expressed in brain tumors, e.g., invasive brain tumors. As such, brain tumors, e.g., invasive brain tumors, can be identified using doublecortin expression as a marker, where an elevated level of doublecortin in a biological sample from the brain of a subject compared to a control subject not having a brain tumor is indicative of the presence of a brain tumor in the subject. The presence, level, or absence of doublecortin in a biological sample from the brain can be determined using, e.g., a doublecortin binding agent. Suitable doublecortin binding agents can include, e.g., a nucleic acid, a polypeptide, e.g., an antibody described herein, a peptide fragment, a peptidomimetic, or a small molecule.

A variety of methods can be used to determine the level of doublecortin protein. In general, these methods include contacting an agent that selectively binds to the protein, such as an antibody, with a biological sample, to evaluate the level of protein in the biological sample. An anti-doublecortin antibody can be, e.g., a polyclonal antibody; a monoclonal antibody or antigen binding fragment thereof; a modified antibody such as a chimeric antibody, reshaped antibody, humanized antibody, or fragment thereof (e.g., Fab′, Fab, F(ab′)₂); or a biosynthetic antibody, e.g., a single chain antibody, single domain antibody (DAB), Fv, single chain Fv (scFv), or the like.

Methods of making and using polyclonal and monoclonal antibodies to detect a particular target are described in, e.g., Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, and F(ab′)₂ fragments), or biosynthetic antibodies (e.g., single chain antibodies, single domain antibodies (DABs), Fv, single chain Fv (scFv), and the like), are known in the art and can be found in, e.g., Zola, Monoclonal Antibodies: Preparation and Use of Monoclonal Antibodies and Engineered Antibody Derivatives, Springer Verlag (2000, 1st edition).

In a preferred embodiment, the antibody bears a detectable label. The term “labeled,” with regard to a probe or antibody, is intended to encompass direct labeling of the probe or antibody by coupling (i.e., physically linking) a detectable substance to the probe or antibody, as well as indirect labeling of the probe or antibody by reactivity with a detectable substance. The antibody can be labeled with, e.g., a fluorescent tag, e.g., GFP, a radioactive tag, a myc tag, or a his tag.

The detection methods can be used to detect doublecortin in a biological sample, e.g., brain tissue or cerebrospinal fluid, in vitro as well as in vivo. In vitro techniques for detection include enzyme linked immunosorbent assays (ELISAs), immunoprecipitation, immunofluorescence, enzyme immunoassay (EIA), radioimmunoassay (RIA), and Western blot analysis. In vivo techniques for detection of doublecortin include introducing a labeled antibody into a subject's brain. For example, the antibody can be labeled with a radioactive marker whose presence and location in a subject's brain can be detected by standard imaging techniques. In another embodiment, the biological sample is labeled, e.g., biotinylated, and then contacted to the antibody, e.g., an antibody positioned on an antibody array. The sample can be detected, e.g., with avidin coupled to a fluorescent label.

The presence, level, or absence of doublecortin in brain tumors can also be evaluated by contacting a biological sample, e.g., brain tissue or cerebrospinal fluid, with a compound or an agent capable of detecting the nucleic acid, e.g., mRNA or genomic DNA, that encodes doublecortin, such that the presence of the nucleic acid is detected. The level of mRNA corresponding to doublecortin in a biological sample, e.g., brain tissue or cerebrospinal fluid, can be determined both by in situ and by in vitro methods.

Isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid probe that can hybridize to the mRNA encoded by the gene being detected. The nucleic acid probe can be, for example, a full-length nucleic acid, or a portion thereof, such as an oligonucleotide of at least 7, 15, 30, 50, 100, 250 or 500 nucleotides in length and sufficient to specifically hybridize under stringent conditions to mRNA or genomic DNA of doublecortin. The probe can be disposed on an address of an array, e.g., an array described below. Other suitable probes for use in the diagnostic assays are described herein.

The nucleic acid probe may further be modified to contain a detectable label for diagnostic purposes. A variety of such labels are known in the art and can readily be employed with the nucleic acid molecules described herein. Suitable labels include, but are not limited to, biotin, radiolabeled nucleotides, and the like. A skilled artisan can employ any of the labels known in the art to obtain a labeled nucleic acid molecule.

In one format, mRNA or cDNA is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA or cDNA is contacted with the probes, for example, in a two-dimensional gene chip array described below. A skilled artisan can adapt known mRNA detection methods for use in detecting the level of mRNA encoded by the gene.

The level of mRNA in a sample that is encoded by a gene can be evaluated with nucleic acid amplification, e.g., by rtPCR (Mullis, U.S. Pat. No. 4,683,202), ligase chain reaction (Barany, Proc. Natl. Acad. Sci. USA, 88:189-193 (1991)), self sustained sequence replication (Guatelli et al., Proc. Natl. Acad. Sci. USA, 87:1874-1878 (1990)), transcriptional amplification system (Kwoh et al., Proc. Natl. Acad. Sci. USA, 86:1173-1177 (1989)), Q-Beta Replicase (Lizardi et al., Bio/Technology, 6:1197 (1988)), rolling circle replication (Lizardi et al., U.S. Pat. No. 5,854,033) or any other nucleic acid amplification method, followed by the detection of the amplified molecules using techniques known in the art. As used herein, amplification primers are defined as a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell, e.g., a brain cell, or tissue sample, e.g., brain tissue sample, can be prepared or processed and then immobilized on a support, typically a glass slide, and contacted with a probe that can hybridize to mRNA that encodes the gene being analyzed, e.g., doublecortin gene.

In another embodiment, serial analysis of gene expression, as described in U.S. Pat. No. 5,695,937, is used to detect transcript levels of doublecortin.

In another embodiment, the methods further include contacting the control sample with a compound or agent capable of detecting doublecortin protein, and comparing the presence of doublecortin protein in the control sample with the presence of the protein in the test sample.

The invention also includes kits for detecting the presence of doublecortin in a biological sample. For example, the kit can include a compound or agent capable of detecting doublecortin protein, e.g., an antibody, or mRNA, e.g., a nucleic acid probe, and a standard. The compound or agent can be packaged in a suitable container. The kit can further include instructions for using the kit to evaluate a subject, e.g., for risk for an invasive brain tumor.

Methods of Screening for Modulators of Doublecortin

The invention provides methods for identifying compounds, e.g., small organic or inorganic molecules (M.W. less than 1,000 Da), oligopeptides, oligonucleotides, or carbohydrates, capable of modulating (i.e., reducing or increasing) doublecortin expression or activity and, therefore, treating a brain tumor.

Libraries of Test Compounds

In certain embodiments, screens of the present invention utilize libraries of test compounds. As used herein, a “test compound” can be any chemical compound, for example, a macromolecule (e.g., a polypeptide, a protein complex, glycoprotein, or a nucleic acid) or a small molecule (e.g., an amino acid, a nucleotide, an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids), amino acids, amino acid analogs, polynucleotides, polynucleotide analogs, nucleotides, nucleotide analogs, and organic or inorganic compounds, e.g., heteroorganic or organometallic compounds.

Test compounds can be screened individually or in parallel. An example of parallel screening is a high throughput drug screen of large libraries of chemicals. Such libraries of candidate compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. Alternatively, prior experimentation and anecdotal evidence can suggest a class or category of compounds of enhanced potential. A library can be designed and synthesized to cover such a class of chemicals.

The synthesis of combinatorial libraries is well known in the art and has been reviewed (see, e.g., Gordon et al., J. Med. Chem., 37:1385-1401 (1994); DeWitt et al., Acc. Chem. Res., 29:114 (1996); Armstrong et al., Acc. Chem. Res., 29:123 (1996); Ellman, Acc. Chem. Res., 29:132 (1996); Gordon et al., Acc. Chem. Res., 29:144 (1996); Lowe, Chem. Soc. Rev., 309 (1995); Blondelle et al., Trends Anal. Chem., 14:83 (1995); Chen et al., J. Am. Chem. Soc., 116:2661 (1994); U.S. Pat. Nos. 5,359,115, 5,362,899, and 5,288,514; PCT Publication Nos. WO92/10092, WO93/09668, WO91/07087, WO93/20242, and WO94/08051).

Libraries of compounds can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (see, e.g., M. Bodansky “Principles of Peptide Synthesis”, 2nd edition, Springer-Verlag, Berlin (1993)). To each aliquot of beads is added a solution of a different activated amino acid, and the reactions are allowed to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with a target protein, e.g., doublecortin. It will be appreciated that a wide variety of peptidic, peptidomimetic, or non-peptidic compounds can be readily generated in this way.

The “split-pool” strategy can result in a library of peptides, e.g., modulators, which can be used to prepare a library of test compounds of the invention. In another illustrative synthesis, a “diversomer library” is created by the method of DeWitt et al. (Proc. Natl. Acad. Sci. U.S.A., 90:6909 (1993)). Other synthesis methods, including the “tea-bag” technique of Houghten (see Nature, 354:84-86 (1991)) can also be used to synthesize libraries of compounds according to the subject invention.

Libraries of test compounds can be screened to determine whether any members of the library have a desired activity, and, if so, to identify the active species. Methods of screening combinatorial libraries have been described (see, e.g., Gordon et al., J. Med. Chem., supra). Soluble test compound libraries can be screened by affinity chromatography with an appropriate target protein to isolate interacting test compounds, followed by identification of the isolated compounds by conventional techniques (e.g., mass spectrometry, NMR, and the like). Immobilized test compounds can be screened by contacting the compounds with a target protein; in some methods, the target protein is conjugated to a label (e.g., fluorophores, colorimetric enzymes, radioisotopes, luminescent compounds, and the like) that can be detected to indicate test compound binding.

Screening Methods

The invention provides methods for identifying compounds capable of modulating doublecortin level or activity. Although applicants do not intend to be bound by any particular theory as to the biological mechanism involved, such compounds are thought to modulate specifically (1) the function of a doublecortin polypeptide and/or (2) expression of the doublecortin gene.

In certain aspects of the present invention, screening for such compounds is accomplished by (i) identifying from a group of test compounds those that bind to a doublecortin polypeptide, and/or modulate (i.e., increase or decrease) transcription and/or translation of doublecortin; and, optionally, (ii) further testing such compounds for their ability to modulate doublecortin activity in vitro or in vivo. Test compounds that bind to doublecortin or modulate transcription and/or translation of doublecortin are referred to herein as “candidate compounds.” Candidate compounds further tested and found to be capable of modulating in vitro or in vivo the activity of a doublecortin polypeptide and/or a brain tumor are considered “doublecortin modulating agents.” In the screening methods of the present invention, candidate compounds can be, but do not necessarily have to be, tested to determine whether they are doublecortin modulating agents. Assays of the present invention may be carried out in whole cell preparations and/or in ex vivo cell-free systems.

In one aspect, the invention includes methods for screening test compounds to identify compounds that bind specifically to doublecortin polypeptides. Binding of a test compound to a doublecortin polypeptide can be detected, for example, in vitro by reversibly or irreversibly immobilizing the test compound(s) on a substrate, e.g., the surface of a well of a 96-well polystyrene microtitre plate. Methods for immobilizing polypeptides and other small molecules are well known in the art. For example, microtitre plates can be coated with a doublecortin polypeptide by adding the polypeptide in a solution (typically, at a concentration of 0.05 to 1 mg/ml in a volume of 1-100 μl) to each well, and incubating the plates at room temperature to 37° C. for a given amount of time, e.g., for 0.1 to 36 hours. Polypeptides not bound to the plate can be removed by shaking excess solution from the plate, and then washing the plate (once or repeatedly) with water or a buffer. Typically, the polypeptide is in water or a buffer. The plate can then be washed with a buffer that lacks the bound polypeptide. To block the free protein-binding sites on the plates, plates can be blocked with a protein that is unrelated to the bound polypeptide. For example, 300 μl of bovine serum albumin (BSA) at a concentration of 2 mg/ml in Tris-HCl can be used. Suitable substrates include those substrates that contain a defined cross-linking chemistry (e.g., plastic substrates, such as polystyrene, styrene, or polypropylene substrates from Corning Costar Corp. (Cambridge, MA), for example). If desired, a beaded particle, e.g., beaded agarose or beaded sepharose, can be used as the substrate. Test compounds can then be added to the coated plate and allowed to bind to the doublecortin polypeptide (e.g., at 37° C. for 0.5-12 hours). The plate can then be rinsed as described above.

Binding of doublecortin to a test compound can be detected by any of a variety of art-known methods. For example, an antibody that specifically binds to a doublecortin polypeptide (i.e., an anti-doublecortin antibody described herein) can be used in an immunoassay. If desired, the antibody can be labeled (e.g., fluorescently or with a radioisotope) and detected directly (see, e.g., West et al., J. Cell Biol., 74:264 (1977)). Alternatively, a second antibody can be used for detection (e.g., a labeled antibody that binds to the Fc portion of the anti-doublecortin antibody). In an alternative detection method, the doublecortin polypeptide is labeled (e.g., with a radioisotope, fluorophore, chromophore, or the like), and the label is detected. In still another method, a doublecortin polypeptide is produced as a fusion protein with a protein that can be detected optically, e.g., green fluorescent protein (that can be detected under UV light). In an alternative method, the polypeptide is produced as a fusion protein with an enzyme having a detectable enzymatic activity, such as horseradish peroxidase, alkaline phosphatase, β-galactosidase, or glucose oxidase. Genes encoding all of these enzymes have been cloned and are available for use by skilled practitioners. If desired, the fusion protein can include an antigen, which can be detected and measured with a polyclonal or monoclonal antibody using conventional methods. Suitable antigens include enzymes (e.g., horse radish peroxidase, alkaline phosphatase, and β-galactosidase) and non-enzymatic polypeptides (e.g., serum proteins, such as BSA and globulins, and milk proteins, such as caseins).

In various methods for identifying polypeptides (e.g., test polypeptides) that bind to a doublecortin polypeptide, the conventional two-hybrid assays of protein/protein interactions can be used (see, e.g., Chien et al., Proc. Natl. Acad. Sci. USA, 88:9578 (1991); Fields et al., U.S. Pat. No. 5,283,173; Fields et al., Nature, 340:245 (1989); Le Douarin et al., Nucleic Acids Research, 23:876 (1995); Vidal et al., Proc. Natl. Acad. Sci. USA, 93:10315-10320 (1996); and White, Proc. Natl. Acad. Sci. USA, 93:10001-10003 (1996)). Generally, two-hybrid methods involve reconstitution of two separable domains of a transcription factor. One fusion protein contains the doublecortin polypeptide fused to either a transactivator domain or DNA binding domain of a transcription factor (e.g., of Gal4). The other fusion protein contains a test polypeptide fused to either the DNA binding domain or a transactivator domain of a transcription factor. Once brought together in a single cell (e.g., a yeast cell or mammalian cell), one of the fusion proteins contains the transactivator domain and the other fusion protein contains the DNA binding domain. Therefore, binding of the doublecortin polypeptide to the test polypeptide reconstitutes the transcription factor. Reconstitution of the transcription factor can be detected by detecting expression of a gene (e.g., a reporter gene) that is operably linked to a DNA sequence that is bound by the DNA binding domain of the transcription factor. Kits for practicing various two-hybrid methods are commercially available (e.g., from Clontech; Palo Alto, Calif.).

In one aspect, the invention provides methods of identifying test compounds that modulate (e.g., increase or decrease) expression of a doublecortin polypeptide. The method includes contacting a doublecortin nucleic acid with a test compound and then measuring expression of the encoded doublecortin polypeptide. In a related aspect, the invention features a method of identifying compounds that modulate (e.g., increase or decrease) the expression of doublecortin polypeptides by measuring expression of a doublecortin polypeptide in the presence of the test compound or after the addition of the test compound in: (a) a cell line into which has been incorporated a recombinant construct including the doublecortin nucleic acid sequence or fragment or an allelic variation thereof; or (b) a cell population or cell line that naturally selectively expresses doublecortin, and then measuring the activity of doublecortin and/or the expression thereof.

Since the doublecortin nucleic acids described herein have been identified, they can be cloned into various host cells (e.g., fungi, E. coli, or yeast) for carrying out such assays in whole cells.

In certain embodiments, an isolated nucleic acid molecule encoding a doublecortin polypeptide is used to identify a compound that modulates (e.g., increases or decreases) the expression of doublecortin in vivo (e.g., in a doublecortin-producing cell). In such embodiments, cells that express doublecortin are cultured, exposed to a test compound (or a mixture of test compounds), and the level of doublecortin expression or activity is compared with the level of doublecortin expression or activity in cells that are otherwise identical but that have not been exposed to the test compound(s). Standard quantitative assays of gene expression and doublecortin activity can be used.

Expression of a doublecortin polypeptide can be measured using art-known methods, for example, by Northern blot PCR analysis or RNAse protection analyses using a nucleic acid molecule of the invention as a probe. Other examples include enzyme-linked immunosorbent assay (ELISA), radioimmunoassay (RIA) and fluorescent activated cell sorting (FACS). The level of expression in the presence of the test molecule, compared with the level of expression in its absence, will indicate whether or not the test compound modulates the expression of doublecortin.

In still another aspect, the invention provides methods of screening test compounds utilizing cell systems that are sensitive to perturbation to one or several transcriptional/translational components. In one embodiment, the cell system is a modified doublecortin-expressing cell in which one or more of the transcriptional/translational components of the cell are present in an altered form or in a different amount compared with a corresponding wild-type doublecortin-expressing cell. This method involves examining a test compound for its ability to perturb transcription/translation in such a modified cell.

In certain embodiments, the methods include identifying candidate compounds that interfere with steps in doublecortin translational accuracy, such as maintaining a proper reading frame during translation and terminating translation at a stop codon. This method involves constructing cells in which a detectable reporter polypeptide can only be produced if the normal process of staying in one reading frame or of terminating translation at a stop codon has been disrupted. This method further involves contacting the cell with a test compound to examine whether it increases or decreases the production of the reporter polypeptide.

In other embodiments, the cell system is a cell-free extract and the method involves measuring transcription or translation in vitro. Conditions are selected so that transcription or translation of the reporter is increased or decreased by the addition of a transcription modifier or a translation modifier to the cell extract.

One method for identifying candidate compounds relies upon a transcription-responsive gene product. This method involves constructing a cell in which the production of a reporter molecule changes (i.e., increases or decreases) under conditions in which cell transcription of a doublecortin nucleic acid changes (i.e., increases or decreases). Specifically, the reporter molecule is encoded by a nucleic acid transcriptionally linked to a sequence constructed and arranged to cause a relative change in the production of the reporter molecule when transcription of a doublecortin nucleic acid changes. A gene sequence encoding the reporter may, for example, be fused to part or all of the gene encoding the transcription-responsive gene product and/or to part or all of the genetic elements that control the production of the gene product. Alternatively, the transcription-responsive gene product may stimulate transcription of the gene encoding the reporter, either directly or indirectly. The method further involves contacting the cell with a test compound, and determining whether the test compound increases or decreases the production of the reporter molecule in the cell.

Alternatively, the method for identifying candidate compounds can rely upon a translation-responsive gene product. This method involves constructing a cell in which cell translation of a doublecortin nucleic acid changes (i.e., increases or decreases). Specifically, the reporter molecule is encoded by nucleic acid either translationally linked or transcriptionally linked to a sequence constructed and arranged to cause a relative increase or decrease in the production of the reporter molecule when transcription of a doublecortin nucleic acid changes. A gene sequence encoding the reporter may, for example, be fused to part or all of the gene encoding the translation-responsive gene product and/or to part or all of the genetic elements that control the production of the gene product. Alternatively, the translation-responsive gene product may stimulate translation of the gene encoding the reporter, either directly or indirectly. The method further involves contacting the cell with a test compound, and determining whether the test compound increases or decreases the production of the first reporter molecule in the cell.

For these and any method described herein, a wide variety of reporters may be used, with typical reporters providing conveniently detectable signals (e.g., by spectroscopy). By way of example, a reporter gene may encode an enzyme that catalyses a reaction that alters light absorption properties. Examples of reporter molecules include, but are not limited to, β-galactosidase, invertase, green fluorescent protein, luciferase, chloramphenicol acetyltransferase, beta-glucuronidase, exo-glucanase and glucoamylase. Alternatively, radiolabeled or fluorescent tag-labeled nucleotides can be incorporated into nascent transcripts that are then identified when bound to oligonucleotide probes. For example, the production of the reporter molecule can be measured by the enzymatic activity of the reporter gene product, such as β-galactosidase.

Any of the methods described herein can be used for high throughput screening of numerous test compounds to identify candidate compounds. By high-throughput screening is meant that the method can be used to screen a large number of candidate compounds relatively easily and quickly, e.g., using mechanized screening devices that handle large numbers of samples, e.g., 64 or 256 or more samples per multi-well plate, and 100s or 1000s of samples per assay.

Having identified a test compound as a candidate compound, the candidate compound can be further tested to confirm whether it is a doublecortin modulating agent, i.e., to determine whether it can modulate doublecortin and/or brain tumor activity in vitro or in vivo (e.g., using an animal, e.g., rodent, model system) if desired. Using other, art-known variations of such methods, one can test the ability of a nucleic acid (e.g., DNA or RNA) used as the test compound to bind doublecortin polypeptides or nucleic acids.

In vitro testing of a candidate compound can be accomplished by means known to those in the art, such as assays involving the use of cell lines, e.g., wild type glioma cell lines and/or transgenic glioma cell lines. For example, a candidate compound can be tested to confirm whether it is a doublecortin modulating agent by assaying the effect of the compound on the invasiveness of a glioma cell line, using an in vitro assay as described in, e.g., (Jung et al., J. Cancer Res. Clin. Oncol., 128:469-76 (2002); Jung et al., J. Neurosurg., 94:80-9 (2001); Soroceanu et al., J. Neurosci., 19:5942-54 (1999); Matsumura et al., Biochem. Biophys. Res. Commun., 269:513-20 (2000)). Glioma cell lines are known in the art and include, e.g., U87MG, U118MG, U138MG, U373MG, A172, LN18, LN229, HS683, and C6.

Alternatively or in addition, in vivo testing of candidate compounds can be performed by means known to those in the art. For example, the candidate compound(s) can be administered to a mammal, such as a rodent (e.g., murine) or rabbit. Such animal model systems are art-accepted for testing potential pharmaceutical agents to determine their therapeutic efficacy in patients, e.g., human patients. Animals that are particularly useful for in vivo testing are wild type animals or non-wild type animals (e.g., mice) that over-produce doublecortin polypeptides, e.g., animals that overexpresses a doublecortin transgene. In a typical in vivo assay, an animal (e.g., a wild type or transgenic mouse) is administered, by any route deemed appropriate (e.g., by injection), a dose of a candidate compound. Conventional methods and criteria can then be used to monitor animals for signs of modulation of doublecortin activity, e.g., signs of increased or decreased brain tumor activity. If needed, the results obtained in the presence of the candidate compound can be compared with results in control animals, which are not treated with the test compound.

Medicinal Chemistry

Once a compound of interest has been identified, standard principles of medicinal chemistry can be used to produce derivatives of the compound. Derivatives can be screened for improved pharmacological properties, for example, efficacy, pharmacokinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry could modify moieties on a candidate compound and measure the effects of the modification on the efficacy of the compound to thereby produce derivatives with increased potency. For an example, see Nagarajan et al., J. Antibiot. 41:1430-8 (1988). Furthermore, if the biochemical target of the compound is known or determined, the structure of the target and the compound can inform the design and optimization of derivatives. Molecular modeling software is commercially available (e.g., Molecular Simulations, Inc.) for this purpose.

Methods of Modulating Doublecortin Level or Activity

Nucleic Acid Molecules

The methods and compositions described herein include the use of nucleic acid molecules, e.g., antisense, ribozyme, and siRNA nucleic acid molecules, that modulate the level or activity of doublecortin by, e.g., targeting a doublecortin gene or mRNA. The nucleic acid molecules include those identified in the methods and assays described herein.

Specifically contemplated are isolated portions of genomic DNA, cDNA, mRNA, antisense, siRNA, and ribozyme nucleic acid molecules, as well as nucleic acid molecules based on an alternative backbone, or nucleic acid molecules containing alternative bases, whether derived from natural sources or synthesized. Such nucleic acid molecules will hybridize under appropriate stringency conditions to a doublecortin gene or mRNA.

The ability of two nucleotide sequences to hybridize to each other is based upon the degree of complementarity of the two nucleotide sequences, which in turn is based on the fraction of matched complementary nucleotide pairs. The more that nucleotides in a given sequence are complementary to another sequence, the greater the degree of hybridization of one to the other. The degree of hybridization also depends on the conditions of stringency, e.g., temperature, solvent ratios, and salt concentrations. In particular, “selective hybridization” pertains to conditions in which the degree of hybridization of a nucleic acid molecule described herein to the target, e.g., doublecortin gene or mRNA, would require complete or nearly complete complementarity. The complementarity must be sufficiently high to ensure that a nucleic acid molecule described herein will bind specifically to the target nucleotide sequence, relative to the binding of other nucleic acids present in the hybridization medium. With selective hybridization, complementarity will be at least 95 to 100%, e.g., 97%.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1994), 6.3.1-6.3.6. Aqueous and nonaqueous methods are described in that reference and either can be used. As used herein, high stringency conditions are 0.5M sodium phosphate, 7% SDS at 65° C., followed by one or more washes in 0.2×SSC, 1% SDS at 65° C.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 85%, 90%, 95% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number and lengths of gaps that need to be introduced for optimal alignment of the two sequences.

The comparison of sequences and determination of percent identity between two sequences can be accomplished using a mathematical algorithm. For example, the percent identity between two amino acid sequences can be determined using, e.g., the algorithm described by Needleman et al. in J. Mol. Biol., 48:444-453 (1970), which has been incorporated into the GAP program in the GCG software package (available at www.gcg.com), using the default parameters, e.g., a Blossum 62 scoring matrix with a gap penalty of 12, a gap extend penalty of 4, and a frameshift gap penalty of 5.

Antisense and ribozyme molecules corresponding to the polypeptide coding or complementary sequence can be prepared. Based upon the sequences known in the art and those disclosed herein, one of skill in the art can easily choose and synthesize any of a number of appropriate antisense nucleic acid molecules for use in accordance with the methods described herein. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

Methods of making antisense nucleic acid molecules that bind to mRNA, form triple helices, or are enzymatically active and cleave RNA and single-stranded DNA (ssDNA) are known in the art. See, e.g., Antisense and Ribozyme Methodology:Laboratory Companion (Ian Gibson, ed., Chapman & Hall, 1997) and Ribozyme Protocols: Methods in Molecular Biology (Phillip C. Turner, ed., Humana Press, Clifton, N.J., 1997).

Antisense nucleic acid molecules described herein can be prepared by routine methods for the synthesis of DNA and RNA molecules. These include techniques for chemically synthesizing oligodeoxyribonucleotides (e.g., by use of an automated DNA synthesizer such as are commercially available from Biosearch, Applied Biosystems, etc.) and oligoribonucleotides (e.g., by solid phase phosphoramide chemical synthesis).

An antisense nucleic acid molecule can be chemically synthesized as described, for example, in Beaucage et al., Tetra. Letts., 22:1859-1862 (1981), and Matteucci et al., J. Am. Chem. Soc., 103:3185 (1981). Antisense nucleic acid molecules can by synthesized using naturally occurring nucleotides or variously modified nucleotides designed to increase the biological stability of the molecules or to increase the physical stability of the duplex formed between the antisense and sense nucleic acids molecules. For example, phosphorothioate, phosphoramidate, and methylphosphonate derivatives of nucleotides can be used (see, e.g., U.S. Pat. Nos. 5,176,996; 5,264,564; and 5,256,775). Acridine substituted nucleotides can also be used. The most widely used modified antisense nucleic acid molecules are phosphorothioates, where one of the oxygen atoms in the phosphodiester bond between nucleotides is replaced with a sulfur atom. These phosphorothioate antisense nucleic acid molecules have greater stability in biological fluids than normal oligos and are preferred antisense nucleic acid molecules. As examples, phosphorothioate oligonucleotides can be synthesized, e.g., by the method of Stein et al. (Nucl. Acids Res., 16:3209 (1988)), and methylphosphonate oligonucleotides can be prepared, e.g., by use of controlled pore glass polymer supports (see, e.g., Sarin et al., Proc. Natl. Acad. Sci. U.S.A., 85:7448-7451 (1988)).

The antisense nucleic acid molecules described herein can also be produced biologically by application of recombinant DNA techniques as described in, e.g., Watson, J. D., et al., Molecular Biology of the Gene, Volumes I and II, Benjamin/Cummings Publishing Co., Inc., Menlo Park, Calif. (1987); Darnell, J. E. et al., Molecular Cell Biology, Scientific American Books, Inc., New York, N.Y. (1986); Lewin, B. M., Genes II, John Wiley & Sons, New York, N.Y. (1985); Maniatis, T. et al. (Molecular Cloning: A Laboratory Manual, Cold Spring Harbor Press, Cold Spring Harbor, N.Y. (1982)); and Sambrook, J. et al. (Molecular Cloning: A Laboratory Manual, 2nd Edition, Cold Spring Harbor Press, Cold Spring Harbor, N.Y. (1989). Techniques for synthesizing such molecules are disclosed by, for example, Wu et al., in Prog. Nucl. Acid. Res. Molec. Biol., 21:101-141 (1978). Procedures for constructing recombinant molecules are disclosed in detail by Sambrook et al. (supra).

Typically, a cDNA of a gene of interest is cloned from a library, e.g., a genomic library. The cDNA, or a portion of the cDNA, is then cloned into an expression vector. An “expression vector” is a vector that (due to the presence of appropriate transcriptional and/or translational control sequences) is capable of expressing a DNA (or cDNA) molecule that has been cloned into the vector and of thereby producing a polypeptide or protein. Expression of the cloned sequences occurs when the expression vector is introduced into an appropriate host cell, e.g., in a particular tissue or organ, such as the brain, in the host (e.g., a mammalian, e.g., human, subject). An appropriate mammalian host cell can be any mammalian cell capable of expressing the cloned sequences. Procedures for preparing cDNA and for producing a genomic library are disclosed by Sambrook et al. (supra).

The cDNA, or a portion of the cDNA, can be cloned into an expression vector in accordance with routine techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. In some methods described herein, antisense vectors are used. An “antisense vector” is a vector that contains a nucleic acid molecule inserted in an antisense orientation (i.e., RNA transcribed from the inserted nucleic acid molecule will be of an antisense orientation relative to the target nucleic acid of interest, e.g., mRNA). Such a nucleic acid molecule is referred to herein as an “antisense oligonucleotide.” Techniques for such manipulations are disclosed by Sambrook et al. (supra).

An antisense vector can be in the form of a recombinant plasmid, phagemid, or attenuated virus. Routine methods can be used to obtain suitable antisense vectors (e.g., Mautino et al., Hum. Gene Ther., 13:1027-37 (2002); Mautino et al., Gene Ther, 9:421-31 (2002); Pachori et al., Hypertension, 39:969-75 (2002)). Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

An antisense vector can include a nucleotide sequence other than an antisense oligonucleotide. For example, the antisense vector can include a nucleotide sequence encoding a reporter protein, e.g., green, yellow, or red fluorescent protein (GFP, YFO, or RFP). Both nucleotide sequences can be operably linked to a single promoter, or they can be operably linked to separate promoters, such that expression of the reporter protein indicates or signals expression of the antisense oligonucleotide.

In vitro studies can be performed to quantify the ability of the antisense oligonucleotide to inhibit gene expression. These studies can utilize controls that distinguish between antisense gene inhibition and nonspecific biological effects of nucleic acid molecules. These studies can also compare levels of the target RNA or protein with that of an internal control RNA or protein. Additionally, results obtained using the antisense oligonucleotide can be compared with those obtained using a control nucleotide sequence. Preferably, the control nucleotide sequence is of approximately the same length as the antisense oligonucleotide and the control nucleotide sequence differs from the antisense oligonucleotide no more than is necessary to prevent specific hybridization of the control nucleotide sequence to the target sequence.

Expression patterns within cells or tissues treated with one or more antisense oligonucleotides can be compared to control cells or tissues not treated with antisense oligonucleotides and the patterns produced can be analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns.

Examples of routine methods of gene expression analysis include DNA arrays or microarrays (Brazma et al., FEBS Lett., 480:17-24 (2000); Celis et al., FEBS Lett., 480:2-16 (2000)), SAGE (serial analysis of gene expression) (Madden et al., Drug Discov. Today, 5:415-425 (2000)), READS (restriction enzyme amplification of digested cDNAs) (Prashar et al., Methods Enzymol., 303:258-72 (1999)), TOGA (total gene expression analysis) (Sutcliffe et al., Proc. Natl. Acad. Sci. U.S.A., 97:1976-81 (2000)), protein arrays and proteomics (Celis et al.; supra; Jungblut et al., Electrophoresis, 20:2100-10 (1999)), expressed sequence tag (EST) sequencing (Celis et al., supra; Larsson et al., J. Biotechnol., 80:143-57 (2000)), subtractive RNA fingerprinting (SuRF) (Fuchs et al., Anal. Biochem., 286:91-98 (2000); Larson et al., Cytometry, 41:203-208 (2000)), subtractive cloning, differential display (DD) (Jurecic et al., Curr. Opin. Microbiol. 3:316-21 (2000)), comparative genomic hybridization (Carulli et al., J. Cell Biochem. Suppl., 31:286-96 (1998)), FISH (fluorescent in situ hybridization) techniques (Going et al., Eur. J. Cancer, 35:1895-904 (1999)) and mass spectrometry methods (reviewed in To, Comb. Chem. High Throughput Screen, 3:235-41 (2000)).

Such expression analysis can be used to determine an optimal concentration of an antisense oligonucleotide, or of an antisense vector, to inhibit gene expression. An optimal concentration can be determined by administering different concentrations of the antisense oligonucleotide and monitoring the effect on gene expression.

Also contemplated is the use of compounds that mediate posttranscriptional gene silencing (PTGS), quelling, and RNA interference (RNAi) to modulate doublecortin level or activity. RNAi is a remarkably efficient process whereby double-stranded RNA (dsRNA, also referred to herein as siRNAs or ds siRNAs, for double-stranded small interfering RNAs) induces the sequence-specific degradation of homologous mRNA in animals and plant cells (Hutvagner et al., Curr. Opin. Genet. Dev., 12:225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell., 10:549-561 (2002); Elbashir et al., Nature, 411:494-498 (2001)), or by micro-RNAs (mRNA), functional small-hairpin RNA (shRNA), or other dsRNAs which are expressed in vivo using DNA templates with RNA polymerase III promoters (Zeng et al., Mol. Cell., 9:1327-1333 (2002); Paddison et al., Genes Dev., 16:948-958 (2002); Lee et al., Nature Biotechnol., 20:500-505 (2002); Paul et al., Nature Biotechnol., 20:505-508 (2002); Tuschl, Nature Biotechnol., 20:440-448 (2002); Yu et al., Proc. Natl. Acad. Sci. USA, 99:6047-6052 (2002); McManus et al., RNA, 8:842-850 (2002); Sui et al., Proc. Natl. Acad. Sci. USA, 99:5515-5520 (2002)). Accordingly, the methods described herein include such molecules that are targeted to a doublecortin RNA.

The nucleic acid molecules or constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is identical or substantially identical to the first strand. The dsRNA molecules of the invention can be chemically synthesized, or can transcribed be in vitro from a DNA template, or in vivo from, e.g., shRNA. The dsRNA molecules can be designed using any method known in the art, for instance, by using the following protocol:
1. Beginning with the AUG start codon, look for AA dinucleotide sequences; each AA and the 3′ adjacent 16 or more nucleotides are potential siRNA targets. siRNAs taken from the 5′ untranslated regions (UTRs) and regions near the start codon (within about 75 bases or so) may be less useful as they may be richer in regulatory protein binding sites, and UTR-binding proteins and/or translation initiation complexes may interfere with binding of the siRNP or RISC endonuclease complex. Thus, in one embodiment, the nucleic acid molecules are selected from a region of the cDNA sequence beginning 50 to 100 nt downstream of the start codon. Further, siRNAs with lower G/C content (35-55%) may be more active than those with G/C content higher than 55%. Thus, in one embodiment, the invention includes nucleic acid molecules having 35-55% G/C content. In addition, the strands of the siRNA can be paired in such a way as to have a 3′ overhang of 1 to 4, e.g., 2, nucleotides. Thus, in another embodiment, the nucleic acid molecules can have a 3′ overhang of 2 nucleotides, such as TT. The overhanging nucleotides can be either RNA or DNA.
2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at www.ncbi.nlm.nih.gov/BLAST.
3. Select one or more sequences that meet your criteria for evaluation.

Further general information about the design and use of siRNA can be found in “The siRNA User Guide,” available on the internet at mpibpc.gwdg.de/abteilungen/100/105/sirna.html.

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The siRNAs used in the methods described herein can be crosslinked siRNA derivatives. Crosslinking can be employed to alter the pharmacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

In some embodiments, 5′ phosphorylated siRNAs are used; in other embodiments, hydroxylated forms can be utilized. See, e.g., Lipardi et al., Cell, 107:297-307 (2001); Boutla et al., Curr. Biol., 11:1776-80 (2001); Djikeng et al., RNA, 7:1522-30 (2001); Elbashir et al., EMBO J., 20:6877-88 (2001); Harborth et al., J. Cell. Sci., 114:4557-65 (2001); Hutvagner et al., Science, 293: 811-3 (2001); and Kalidas et al., Neuron, 33:177-184 (2002).

Antibodies

Polyclonal and monoclonal antibodies and fragments of these antibodies that bind to doublecortin can be prepared using methods known in the art. For example, suitable host animals can be immunized using appropriate immunization protocols and the peptides, polypeptides, or proteins described herein. Peptides for use in immunization are typically about 8 to 40 amino acid residues long. If necessary or desired, the polypeptide immunogens can be conjugated to suitable carriers. Methods for preparing immunogenic conjugates with carriers such as bovine serum albumin (BSA), keyhole limpet hemocyanin (KLH), or other carrier proteins are well known in the art (See, e.g., Harlow et al., 1998, supra). In some circumstances, direct conjugation using, for example, carbodiimide reagents, may be effective; in other instances linking reagents such as those supplied by Pierce Chemical Co. (Rockford, Ill.) may be desirable to provide accessibility to the polypeptide or hapten. The hapten peptides can be extended at either the amino or carboxyl terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

Antigenic proteins or polypeptides for use as immunogens can be prepared synthetically in a protein synthesizer and optionally coupled to a carrier molecule and injected over several months into rabbits. Rabbit sera is tested for immunoreactivity. Monoclonal antibodies can be made by injecting mice with doublecortin proteins, or antigenic fragments thereof. Monoclonal antibodies can be screened by ELISA and tested for specific immunoreactivity. (See, e.g., Harlow et al., 1988 supra; and Using Antibodies: A Laboratory Manual, Harlow and Lane, Eds., Cold Spring Harbor Press (1999)). These antibodies are useful in assays and as pharmaceuticals and diagnostics.

Thus, anti-peptide antibodies can be generated using synthetic peptides, for example, peptides derived from the sequence of doublecortin from human or other species. Synthetic antigenic peptides can be as small as 2-3 amino acids in length, but are typically at least about 3, 5, 10, or 15 or more amino acid residues long (or any range in between). Such peptides can be determined using programs such as DNAStar. The peptides can be coupled to KLH using standard methods and can be immunized into animals such as rabbits. Polyclonal peptide antibodies can then be purified, for example using Actigel beads containing the covalently bound peptide.

While the polyclonal antisera produced in this way may be satisfactory for some applications, for pharmaceutical compositions, monoclonal preparations are typically used. Immortalized cell lines that secrete the desired monoclonal antibodies can be prepared using the standard method of Kohler and Milstein or modifications that effect immortalization of lymphocytes or spleen cells, as is generally known (See, e.g., Harlow et al., 1988 and 1999, supra). The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal antibodies that contain the immunologically significant portion can be used as agonists or antagonists of doublecortin activity. In some embodiments, the use of immunologically reactive (i.e., antigen binding) fragments, such as Fab, scFv, Fab′, and F(ab′)₂ fragments, is preferable, especially in a therapeutic context, as these fragments are generally less immunogenic than the whole immunoglobulin.

The antibodies or fragments can also be produced by recombinant means. Antibodies that bind specifically to the desired regions of doublecortin can also be produced in the context of chimeras with multiple species origin. Immunoglobulin reagents so created are contemplated for use diagnostically or as inhibitors of doublecortin level or activity.

In one embodiment, antibodies or antigen-binding fragments bind doublecortin with high affinity, i.e., ranging from 10⁻⁵ to 10⁻⁹ M. In some embodiments, the antibody or antigen-binding fragment will comprise or be derived from (e.g., a portion of) a chimeric, primate, Primatized®, human or humanized antibody.

Another embodiment contemplates chimeric antibodies that recognize doublecortin. A chimeric antibody can be, e.g., an antibody with non-human variable regions and human constant regions, most typically rodent variable regions and human constant regions.

A Primatized® antibody refers to an antibody with primate variable regions, e.g., CDR's, and human constant regions. In some embodiments, such primate variable regions are derived from an Old World monkey.

A humanized antibody refers to an antibody with framework and constant regions that are substantially human, and complementarity-determining regions (CDRs) that are non-human. “Substantially” refers to the fact that humanized antibodies typically retain at least several donor framework residues (i.e., of non-human parent antibody from which CDRs are derived).

Methods for producing chimeric, primate, Primatized®, humanized and human antibodies are well known in the art. See, e.g., Queen et al., U.S. Pat. No. 5,530,101; Winter et al., U.S. Pat. No. 5,225,539; Boss et al., U.S. Pat. No. 4,816,397; and Cabilly et al., U.S. Pat. No. 4,816,567, all of which are incorporated herein by reference in their entirety for all purposes.

The selection of human constant regions may be significant to the therapeutic efficacy of the subject anti-doublecortin antibody. In some embodiments, the subject anti-doublecortin immunoglobulin will comprise human γ₁, or γ₃ constant regions. In some embodiments, the subject anti-doublecortin immunoglobulin will comprise human γ₁ constant regions.

Methods for making human immunoglobulins are also known and include, by way of example, production in SCID mice, and in vitro immunization.

For example, antibodies to doublecortin proteins or peptides can be prepared in the following fashion. cDNAs can be expressed as six-histidine fusion proteins in E. coli, and purified using nickel chelate chromatography, or expressed as GST-fusion proteins purified using glutathione beads. Purified proteins can be injected into rabbits and antisera generated. Anti-peptide antisera can also be generated using techniques known in the art. Antibodies can be affinity purified if necessary.

Human brain libraries can be used to screen for doublecortin-interacting proteins. Immunocytochemistry can be used to determine if interacting proteins co-localize with doublecortin in brain tumors.

Gene Therapy

A nucleic acid molecule described herein, e.g., a nucleic acid molecule encoding a doublecortin-interacting protein, can be incorporated into a gene construct to be used as part of a gene therapy protocol to deliver, e.g., the doublecortin-interacting protein. The methods described herein include the use of expression vectors for in vivo transfection of a nucleic acid molecule described herein. Expression vectors may be administered in any biologically effective carrier, e.g., any formulation or composition capable of effectively delivering the nucleic acid molecule to cells in vivo. To prepare a nucleic acid molecule for administration, the nucleic acid molecule can be suspended in a medium to facilitate transfection into cells using routine techniques. For example, the nucleic acid molecule can be suspended in artificial cerebrospinal fluid and combined with a transfection material, e.g., a lipid, e.g., DOTAP.

Another approach includes insertion of the nucleic acid molecule into viral vectors including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A useful approach for in vivo introduction of a nucleic acid molecule into a cell is the use of a viral vector containing a nucleic acid molecule, e.g., a cDNA. Infection of cells with a viral vector has the advantage that a large proportion of the targeted cells can receive the nucleic acid molecule. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous nucleic acid molecules in vivo, particularly into humans. These vectors provide efficient delivery of nucleic acid molecules into cells, and the transferred nucleic acids are stably integrated into the chromosomal DNA of the host. The development of specialized cell lines (termed “packaging cells”) that produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions that can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals.

Examples of suitable retroviruses include pLJ, pZIP, pWE and pEM, which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see, e.g., Eglitis et al., Science, 230:1395-1398 (1985); Danos et al., Proc. Natl. Acad. Sci. USA, 85:6460-6464 (1988); Ferry et al., Proc. Natl. Acad. Sci. USA, 88:8377-8381 (1991); Chowdhury et al., Science, 254:1802-1805 (1991); Kay et al., Human Gene Therapy, 3:641-647 (1992); Dai et al., Proc. Natl. Acad. Sci. USA, 89:10892-10895 (1992); Hwu et al., J. Immunol., 150:4104-4115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful for the methods described herein utilizes adenovirus-derived vectors. The genome of an adenovirus can be manipulated such that it encodes and expresses a gene product of interest, but is inactivated in terms of its ability to replicate in a normal lytic viral life cycle. See, for example, Berkner et al., BioTechniques, 6:616 (1988); Rosenfeld et al., Science, 252:431-434 (1991); and Rosenfeld et al., Cell, 68:143-155 (1992). Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand et al., J. Virol., 57:267 (1986)).

Yet another viral vector system useful for the methods described herein is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics Micro. Immunol., 158:97-129 (1992)). It is also one of the few viruses that may integrate its DNA into non-dividing cells, and exhibits a high frequency of stable integration (see, e.g., Flotte et al., Am. J. Respir. Cell. Mol. Biol., 7:349-356 (1992); Samulski et al., J. Virol., 63:3822-3828 (1989); and McLaughlin et al., J. Virol., 62:1963-1973 (1989)). Vectors containing as little as 300 base pairs of AAV can be packaged and can integrate. Space for exogenous DNA is limited to about 4.5 kb. An AAV vector such as that described in Tratschin et al., Mol. Cell. Biol., 5:3251-3260 (1985) can be used to introduce DNA into cells. A variety of nucleic acids have been introduced into different cell types using AAV vectors (see, e.g., Hermonat et al., Proc. Natl. Acad. Sci. USA, 81:6466-6470 (1984); Tratschin et al., Mol. Cell. Biol., 4:2072-2081 (1985); Wondisford et al., Mol. Endocrinol., 2:32-39 (1988); Tratschin et al., J. Virol., 51:611-619 (1984); and Flotte et al., J. Biol. Chem., 268:3781-3790 (1993)).

In addition to viral transfer methods, such as those illustrated above, non-viral methods can also be employed to cause expression of a nucleic acid agent described herein (e.g., a nucleic acid molecule encoding a doublecortin-binding polypeptide) in the tissue of a subject.

Administration

Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol., 116:131-135 (2001); Cohen et al., Gene Ther., 7:1896-905 (2000); and Tam et al., Gene Ther., 7:1867-74 (2000).

In a representative embodiment, a gene encoding an agent described herein can be entrapped in liposomes bearing positive charges on their surface (e.g., lipofectins) and (optionally) that are tagged with antibodies against cell surface antigens of the target tissue (see, e.g., Mizuno et al., No Shinkei Geka, 20:547-551 (1992); PCT publication WO91/06309; Japanese patent application 1047381; and European patent publication EP-A-43075).

In clinical settings, the gene delivery systems for the therapeutic gene can be introduced into a patient by any of a number of known methods. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see, e.g., U.S. Pat. No. 5,328,470) or by stereotactic injection (see, e.g., Chen et al., Proc. Natl. Acad. Sci. USA, 91:3054-3057 (1994)) into a specific brain region.

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g., retroviral vectors, the pharmaceutical preparation can comprise one or more cells that produce the gene delivery system.

In some embodiments, a nucleic acid molecule described herein is administered to a subject, e.g., to a brain region of the subject. For injection into the brain, known imaging and stereotactic equipment can be used. The imaging can be performed using direct visualization with a surgical microscope, or can be performed using a camera and/or a computer. The nucleic acid molecules described herein can be delivered into the brain by any routine physical method of introducing material into the brain parenchyma, an anatomical region of the CNS, or the cerebrospinal fluid. Such methods include, e.g., viral delivery, e.g., viral delivery described herein, targeted delivery using liposomes, and direct injection using a miniosmotic pump, a needle, a syringe, or similar mechanism. For example, the nucleic acid molecule can be injected using a needle, e.g., a 30-gauge needle, and a syringe, e.g., a 10 μL syringe.

### EXAMPLES

### Example 1

### Doublecortin Expression in Mouse Brain and in Human Brain Tumors

Because DCX appears essential for migration of neural cells, we suspected that it might have a role in migration of tumor cells and, thus, invasion of neuroepithelial tumors. We hypothesized that tumors with increased DCX expression would be more likely to be invasive.

Tissue Collection

Mouse brains were collected from adult mice, kept in zinc formalin fixative (Anatech Inc., Battle Creek, Mich.) for one week and then embedded in paraffin. Sixty-nine human brain tumors were selected. All specimens had been fixed in 10% buffered formalin and routinely processed through a VIP Tissue-Tek® processor. The original hematoxylin-eosin stained sections from all cases were reviewed and representative sections were chosen from each case for DCX immunohistochemistry. All tumors were classified and graded according to the most recent WHO classification (Kleihues et al., Tumors of the Nervous System. International Agency for Research on Cancer, Lyon (2000)).

Immunohistochemistry

Five μm sections from paraffin-embedded specimens were deparaffinized in xylene, rehydrated in graded alcohol solutions and subjected to antigen retrieval by microwaving twice for 5 minutes in 10 mM sodium citrate, pH 6.0. Endogenous peroxidase activity was quenched with 1% H₂O₂ for 10 minutes. DCX was detected using goat polyclonal anti-DCX antibody N-19 or C-18 (Santa Cruz Biotechnology, Santa Cruz, Calif.) at 1:150 dilution (1.3 μg/ml), incubating slides at room temperature overnight, followed by an anti-goat IgG biotinylated secondary antibody (Vector laboratories, Burlingame, Calif., 7 μg/ml) at room temperature for 2 hours. The sections were washed and incubated with an avidin-horseradish peroxidase complex (Vector Laboratories). Immunoreactivity was detected using a 3,3′-diaminobenzidine kit (Vector laboratories), and sections were counterstained with hematoxylin.

As a control, the anti-DCX antibody was incubated at room temperature for 2 hours with or without the DCX peptide (13.3 μg/ml, Santa Cruz Biotechnology) that was used as immunogen. Then the antibodies with and without peptide were added to adjacent tumor sections. The remainder of the staining procedure was the same as already described.

The DCX-stained slides were evaluated for staining intensity, cellular localization of staining and presence of any distinctive patterns of staining. Intensity of immunoreactivity was evaluated using a semiquantitative scale, where 0=none or same as background, 1=weak (trace or slightly perceptible above background), 2=moderate, and 3=strong (same as positive control). In addition, the percentage of DCX-positive cells for each case was scored as 0, 25, 50, 75 or 100%. Reliability was achieved by having three investigators independently rate the staining intensities. Concordance among the observers was found to be high; interobserver differences were resolved by consensus. The results were statistically analyzed by the Cochran-Mantel-Haenszel statistical test (Mantel, J. Am. Statistical Assoc., 58:690-700 (1963)).

Results

As a first step toward measuring DCX expression, we prepared paraffin sections from healthy adult mouse brains and stained them with anti-DCX N-19 and C-18 antibodies (data not shown). Most regions of the adult mouse brain were DCX-negative. Positive cells were noted in the subventricular zone and a few positive neurons were detected in the hippocampus. These results are consistent with the published results for DCX expression (see Brown et al., J. Comp. Neurol., 467:1-10 (2003); Nacher et al., Eur. J. Neurosci., 14:629-644 (2001); Steiner et al., Glia, 46:41-52 (2004); and Yang et al., J. Neurosci. Res., 76:282-295 (2004)) and provide convincing evidence that the staining procedure used accurately measured the distribution of DCX protein.

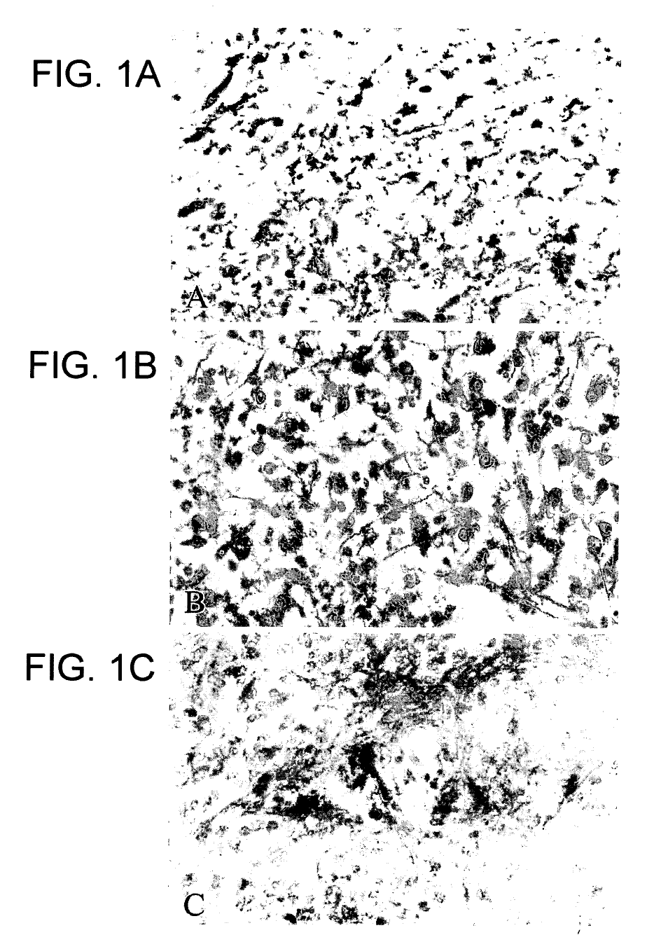

We next stained sections from 11 glioblastomas and found that all of these tumors were strongly positive for DCX (FIG. 1A). This demonstrates that the most invasive type of brain tumor strongly expresses a molecule associated with migrating precursor cells. Given this result, we expanded our study to include a variety of brain tumors. Many invasive tumors, including anaplastic astrocytomas, astrocytomas, oligoastrocytomas and oligodendrogliomas, showed strong staining for DCX (FIG. 1B). Table 1 lists the ranges of staining intensity and percentage of DCX positive cells for each tumor group. Also, pilocytic astrocytomas and other circumscribed tumors showed low-intensity DCX immunoreactivity (FIG. 1C; Table 1).

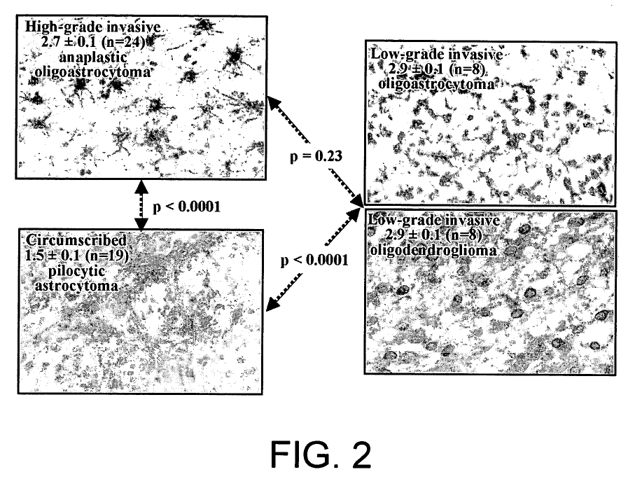

To analyze these data, we pooled the tumor samples into three groups: High-grade invasive (WHO grades III and IV), Low-grade invasive (WHO grade II), and Circumscribed (WHO grade I) (see Table 1 for the assignment of tumor types to these three groups. These assignments were based on the known phenotypic behaviors of these tumor types). We scored the tumors for staining intensity, following a grading scheme commonly used for immunohistochemistry (0, no staining; 1, weak staining; 2, moderate staining; 3, strong staining). By the Cochran-Mantel-Haenszel statistical test (Mantel, J. Am. Statistical Assoc., 58:690-700 (1963)), the circumscribed group was significantly different from both the high-grade invasive group (p<0.0001) and the low-grade invasive group (p<0.0001). The high-grade invasive and low-grade invasive groups were also significantly different (p<0.0001) from the circumscribed tumor group in which the nonglioma tumors (meningiomas and schwnannomas) were omitted. The low-grade invasive and high-grade invasive groups were not significantly different from each other (p=0.23) (see FIG. 2). Hence, although many brain tumors express DCX, staining is significantly greater for invasive tumors.

We next examined DCX levels within the same tumor, comparing the tumor center and margins. For three glioblastomas and one anaplastic oligoastrocytoma, the samples allowed a clear comparison. In each case, DCX staining was clearly greater at the margin than the center.

To confirm the specificity of the DCX immunostaining in human tumors, we carried out four control experiments. First, controls lacking primary anti-DCX antibody were uniformly negative. Second, using a second anti-DCX antibody (Santa Cruz Biotechnology, C-18), we stained sections of a subset of the human brain tumors already stained with the N-19 antibody (1 glioblastoma, 1 anaplastic oligoastrocytoma, 1 schwannoma, and 1 meningioma). The DCX immunostaining obtained with the C-18 antibody was the same as that with the N-19 antibody. Third, we pre-incubated the antibodies with or without the immunizing peptide and then stained adjacent sections. For both N-19 and C-18 anti-DCX antibodies, the immunizing peptides greatly reduced immunostaining of a GBM. In addition, the immunizing peptides also inhibited immunostaining of adult mouse brain, an anaplastic astrocytoma, a schwannoma and a meningioma. Fourth, normal brain adjacent to tumors was negative for DCX staining. Also, one sample of cortex from the temporal lobe of an epileptic patient was stained and found to be negative for DCX. These control experiments demonstrate that we are accurately detecting the distribution of DCX protein in human brain tumors.

TABLE 1
DCX expression in human tumors. Tumors were scored for
staining intensity (0, no staining; 1, weak staining;
2, moderate staining; 3, strong staining).

| | | |
|---|---|---|
| Tumor Type | Mean DCX Staining | Range of % of DCX |
| (Number of samples) | Intensity (Range) | Positive Cells |

HIGH GRADE INVASIVE TUMORS (2.7 ± 0.1, MEAN ± SEM)

| | | |
|---|---|---|
| Glioblastoma multiforme (11) | 2.8 (2-3) | 75-100 |
| Anaplastic astrocytoma (1) | 3 | 75 |
| Anaplastic | 2.0 (1-3) | 50-100 |
| oligoastrocytoma (6) | | |
| PNET (1) | 2 | 100 |
| Medulloblastoma (5) | 2.8 (2-3) | 75-100 |

LOW GRADE INVASIVE TUMORS (2.9 ± 0.1)

| | | |
|---|---|---|
| Astrocytoma (Grade II)(1) | 2 | 100 |
| Oligodendroglioma (3) | 3.0 (3) | 100 |
| Oligoastrocytoma (4) | 3.0 (3) | 100 |

CIRCUMSCRIBED (1.4 ± 0.1)

| | | |
|---|---|---|
| Pilocytic astrocytoma (6) | 1.7 (1-2) | 50-100 |
| Ganglioglioma (2) | 1.5 (1-2) | 25-50 |
| Ependymoma (4) | 1.7 (1-3) | 25-75 |
| Myxopapillary | 1 | 75 |
| ependymoma (1) | | |
| Subependymoma (2) | 1.5 (1-2) | 50-75 |
| DNT (4) | 1.0 (1) | 25 |
| Meningioma (grade I)(9) | 1.2 (1-2) | 25-100 |
| Schwannoma (9) | 1.2 (1-2) | 75 |

### Example 2

### Doublecortin Expression in a Glioma Cell Line

Cell Culture

A U373 human glioma line was cultured in Dulbecco's modified Eagle's medium/high glucose and L-glutamine (Gibco, Carlsbad, Calif.) supplemented with 10% fetal bovine serum, and maintained in a 37° C. incubator with 5% CO₂.

Immunofluorescence Microscopy

U373 cells were grown on glass coverslips, fixed with methanol for 12 minutes at −20° C. and then blocked with PBS+2.5% normal horse serum for 1.5 hours at 37° C. The cells were incubated with N-19 anti-DCX antibody (1:50) for 1.5 hours at 37° C. After washing, the cells were incubated with rhodamine goat anti-rabbit antibody (Jackson Immunosearch, West Grove, Pa., 1:50) and washed. The coverslips were mounted using Vectashield with DAPI (Vector Laboratory, Burlingame, Calif.).

Western Blot Analysis

Total protein was extracted with RIPA buffer (Boston Bioproducts, Ashland, Mass.), 50 mM Tris-HCl, pH 7.4, 150 mM NaCl, 1% NP-40, 0.5% sodium deoxycholate, 0.1% SDS, and 5 mM EDTA, supplemented with 1 mM phenylmethylsulfonyl fluoride (PMSF), and a protease inhibitor cocktail (PIC) (1:200, Sigma Chemicals, St. Louis, Mo.). Total extracts were run on a 10% SDS-PAGE gel and transferred to BioTrace PVDF membranes (Pall Corp., Pensacola, Fla.). Blots were blocked in 2.5% normal rabbit serum in TBS with 0.05% Tween 20 (TBS-T) for one hour and then probed with either anti-DCX N-19 or C-18 antibody (Santa Cruz Biotechnology) at 1:500 dilution in 2.5% NRS in TBS-T for 2.5 hours at room temperature. A rabbit anti-goat IgG peroxidase conjugated (Jackson Immunoresearch) secondary antibody (1:5,000) was applied for 2.5 hours at room temperature. After washing, immunoreactivity was detected by chemiluminescence.

Results

We assessed glioma U373 cells for DCX protein. By immunofluorescence microscopy with the N-19 anti-DCX antibody, DCX showed a punctate distribution in the cytoplasm. A control lacking primary antibody, there was no significant staining. By Western blotting with both the N-19 and C-18 anti-DCX antibodies, the molecular weight of U373 DCX is 40,000 Da (data not shown), consistent with a previous measurement of DCX in the developing brain (Francis et al., Neuron, 23:247-256 (1999)). These results indicate that the U373 cell line can be used for methods, e.g., in vitro methods, described herein.

### Example 3

### Diagnosis of a Brain Tumor by Analyzing Doublecortin Expression

A brain biopsy is obtained from a subject for diagnostic analysis of a brain tumor. The brain biopsy is obtained from the subject under anesthesia by drilling a small hole into the skull, and inserting a needle into the brain tissue guided by computer-assisted imaging techniques (CT or MRI scans). After collection, the brain tissue is fixed in 10% buffered formalin by immersion and embedded in paraffin. The paraffin blocks are cut into 5 μm sections, and analyzed as described in Example 1. The presence and level of DCX expression correlates with the presence of a brain tumor and the relative invasiveness of the brain tumor.

Cerebrospinal fluid (CSF) is obtained through a lumbar puncture, or spinal tap. CSF is obtained by cisternal puncture in lateral recumbency during general anesthesia. After surgical preparation, the puncture of the cerebellomedullary cistern is performed between the occipital bone and the atlas using 22 gauge, 1.5-inch spinal needles with a stylet. For the procedure, the head is held at a right angle to the vertebral column. The needle is carefully inserted, and when the stylet is removed, CSF is collected into a sterile tube.

CSF (10 μl) is mixed with 10 μl of 2× sample loading buffer (1×50 mM Tris-HCl [pH 6.8], 100 mM dithiothreitol, 2% sodium dodecyl sulfate [SDS], 0.1% bromophenol blue, 10% glycerol), heated for 10 min at 100° C., separated by SDS-15% polyacrylamide gel electrophoresis (SDS-15% PAGE), and then transferred to Immobilon polyvinylidene difluoride (PVDF) membranes (Millipore Corp.). Membranes are incubated with anti-DCX goat antibody (SC-8067 or SC-8066, Santa Cruz Biotechnology, polyclonal, affinity-purified antibodies, 1 μg/ml) in phosphate-buffered saline containing 0.2% Tween 20. After washing, bound antibodies are detected by anti-goat IgG (1:5,000) conjugated with horseradish peroxidase (Amersham Pharmacia) followed by chemiluminescence (ECL; Amersham Pharmacia). Detection of doublecortin in the CSF is a positive diagnosis for a brain tumor.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of diagnosing a brain tumor in a subject, the method comprising:
(a) obtaining a biological sample from the brain or cerebrospinal fluid of the subject; and
(b) determining the level or activity of doublecortin in the biological sample, wherein an elevated level or activity of doublecortin in the sample compared to a control indicates the presence of a brain tumor in the subject.

2. The method of claim 1, wherein the sample is cerebrospinal fluid.

3. The method of claim 1, wherein determining comprises using a doublecortin-specific antibody.

4. The method of claim 3, wherein the antibody is labeled.

5. The method of claim 1, further comprising determining a level or activity of doublecortin compared to a threshold, wherein a level or activity above the threshold indicates that the brain tumor is an invasive brain tumor.

6. The method of claim 5, wherein the threshold is the level or activity of doublecortin in a biological sample from the brain or cerebrospinal fluid of a control subject not having a brain tumor.

7. The method of claim 5, wherein the threshold is the level or activity of doublecortin in a biological sample from a noninvasive brain tumor.

8. A method of identifying a candidate compound for the treatment of an invasive brain tumor, the method comprising:
providing a sample comprising doublecortin, or a fragment thereof;
contacting the sample with one or more test compounds; and
evaluating the interaction of the one or more test compounds with doublecortin, wherein a test compound that interacts with doublecortin is a candidate compound for the treatment of an invasive brain tumor.

9. The method of claim 8, wherein the test compound is a small molecule, a peptide, or a nucleic acid.

10. The method of claim 8, further comprising testing whether the test compound selectively reduces the level or activity of doublecortin in vivo.

11. The method of claim 10, wherein testing comprises:
(a) administering the test compound to the subject;
(b) obtaining a biological sample from the brain or cerebrospinal fluid of the subject; and
(c) determining the level or activity of doublecortin in the biological sample, wherein a reduced level or activity of doublecortin in the sample compared to the level or activity before the administration indicates the test compound selectively reduces the level or activity of doublecortin in vivo.

12. A method of treating a subject, the method comprising administering to a subject having or at risk for developing an invasive brain tumor, an agent that selectively reduces the level or activity of doublecortin in an amount sufficient to treat or inhibit formation of an invasive brain tumor in the subject.

13. The method of claim 12, wherein the agent is a nucleic acid construct comprising a nucleotide sequence that is complementary to a portion of a doublecortin mRNA in an amount effective to inhibit translation of the doublecortin mRNA.

14. The method of claim 12, further comprising identifying a subject having or at risk for developing an invasive brain tumor, the method comprising:
(a) obtaining a biological sample from the brain or cerebrospinal fluid of the subject; and
(b) evaluating the biological sample for doublecortin expression, wherein elevated doublecortin expression in the biological sample compared to a control is indicative of a risk for developing an invasive brain tumor.

15. The method of claim 14, wherein the biological sample is cerebrospinal fluid.
